(19) United States
(12) Patent Application Publication
(10) Pub. No.: US 2020/0155443 A1
(43) Pub. Date: May 21, 2020

(54) COSMETIC COMPOSITION FOR WHITENING WITH GREEN TOMATO EXTRACTS, BURDOCK ROOT EXTRACTS AND CHICKPEA EXTRACTS

(71) Applicant: SJ INTERNATIONAL CO., LTD, Chuncheon-si (KR)

(72) Inventor: Yong Hwan OH, Chuncheon-si (KR)

(73) Assignee: SJ INTERNATIONAL CO., LTD, Chuncheon-si (KR)

(21) Appl. No.: 16/685,488

(22) Filed: Nov. 15, 2019

(30) Foreign Application Priority Data
Nov. 15, 2018 (KR) 10-2018-0140543

(51) Int. Cl. A61K 8/97; A61Q 19/02

(52) U.S. Cl. CPC A61K 8/97; A61K 2800/5922; A61Q 19/02

(57) ABSTRACT

A cosmetic composition for skin whitening containing, as an active ingredient, a combination of a green tomato extract, a burdock root extract and a chickpea extract is disclosed.

### BACKGROUND OF THE INVENTION

### Field of the Invention

The present invention relates to a cosmetic composition having an effect of skin whitening containing, as an active ingredient, a combination including a green tomato extract, a burdock root extract and a chickpea extract.

### Description of the Related Art

Skin is an organ that directly receives external stimuli, protects the body, regulates body temperature, evaporation of water and the like, and performs touch perception, skin breathing and the like. In addition, the skin has a cosmetic aspect, so consumers are highly interested in skin care to maintain healthy and elastic skin, which results in active growth of the skin product market.

The Korean cosmetic market grew at an average annual growth rate of 6.7% from 2010 to 2014, and functional cosmetic products for improving whitening and wrinkle and the like have shown an average annual production increase of 18.3% from 2010, and 33.2% of the total cosmetic market is related to functional cosmetic products. The main raw materials of functional cosmetic products are natural materials, organic synthetic substances, polymer compounds and the like, and researches on whitening, anti-aging and the like using these raw materials are being actively conducted.

Meanwhile, consumers prefer skin care products with an enhanced skin whitening effect in order to prevent the phenomenon in which pigmentation readily occurs as skin aging progresses. “Skin whitening” means restoring the clarity of the original skin by inhibiting the unnecessary release or production of a melanin pigment, and refers to making the skin tone white and bright. Recently, there is growing interest in cosmetics that are effective in skin whitening without irritation because they are produced from natural materials and thus are nontoxic to the skin or body. There are conventional technologies relating to whitening cosmetic compositions using natural ingredients such as Korean Patent No. 10-463081 (whitening cosmetic composition containing mung bean extract), Korean Patent No. 10-0829831 (whitening cosmetic composition containing plant extract), Korean Patent Laid-open No. 10-1999-0085941 (whitening cosmetic composition containing grape extract) and the like.

### SUMMARY OF THE INVENTION

Therefore, the present invention has been made in view of the above problems, and it is an object of the present invention to discover extracts of natural products having an excellent skin-whitening effect, and develop and provide a cosmetic composition containing, as an active ingredient, a combination of the extracts.

In accordance with the present invention, the above and other objects can be accomplished by the provision of a cosmetic composition for skin whitening containing, as an active ingredient, a combination of a green tomato extract, a burdock root extract and a chickpea extract.

Meanwhile, in an embodiment of the present invention, the combination of the green tomato extract, the burdock root extract and the chickpea extract preferably inhibits the production of melanin in cells.

Meanwhile, in an embodiment of the present invention, the combination of the green tomato extract, the burdock root extract and the chickpea extract preferably inhibits tyrosinase enzyme activity.

Meanwhile, in an embodiment of the present invention, the combination of the green tomato extract, the burdock root extract and the chickpea extract preferably inhibits DOPA oxidation.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing the result of an experiment to determine the effect of a green tomato extract on the dihydroxyphenylalanine (DOPA) oxidation inhibitory activity;

FIG. 2 is a graph showing the result of an experiment to determine the effect of a chickpea extract on tyrosinase inhibitory activity in cells;

FIG. 3 is a graph showing the result of the effect of a burdock root extract on the production of melanin in cells;

FIG. 4 is a graph showing the results of the effect of a combination of a green tomato extract, a burdock root extract and a chickpea extract on the tyrosinase inhibitory activity in cells;

FIG. 5 is a graph showing the result of an experiment to determine the effect of the combination of the green tomato extract, the burdock root extract and the chickpea extract on the production of melanin in cells;

FIG. 6 is a graph showing the result of an experiment to determine the effect of the combination of the green tomato extract, the burdock root extract and the chickpea extract on tyrosinase inhibitory activity; and

FIG. 7 is a graph showing the result of an experiment to determine the effect of the combination of the green tomato extract, the burdock root extract and the chickpea extract on the inhibitory activity of dihydroxyphenylalanine (DOPA) oxidation.

### DETAILED DESCRIPTION OF THE INVENTION

Reference will now be made in detail to the preferred embodiments of the present invention, examples of which are illustrated in the accompanying drawings. Wherever possible, the same reference numbers will be used throughout the drawings to refer to the same or like parts.

The skin color is determined by the amount of melanin, which is a dark brown pigment present in the basal layer of the epidermis, which is an important factor in skin whitening. Melanin is biosynthesized in the basal layer of the skin, is transported to keratinocytes and migrates to the outer layer of the skin by keratinization. Melanin is produced by a series of enzymatic reactions of tyrosinase, tyrosinase-related protein-1 (TRP-1), tyrosinase-related protein-2 (TRP-2) and the like.

Tyrosinase is an enzyme that catalyzes the initial stage of melanin biosynthesis, which is a step of hydroxylating tyrosine to form dihydroxyphenylalanine (DOPA), and further oxidizing the same to form dopaquinone. Tyrosinase is eventually converted to eumelanin and pheomelanin. Melanin protects the skin from UV rays, external exposure, aging and the like, but excessive production of melanin leads to deposition of pigments in the skin to form spots and freckles. Therefore, research to realize skin anti-aging and whitening by inhibiting the production of melanin by blocking UV rays or protecting cells from ROS has been conducted.

In one aspect, the present invention provides a cosmetic composition for skin whitening containing, as an active ingredient, a combination including a green tomato extract, a burdock root extract and a chickpea extract.

Green tomato is also called “fresh tomato” and is a green tomato that is eaten when the fruit is not completely ripe. Green tomato is hard and astringent, and is a popular tomato in the United States, taken in combination with many types of foods and served in various ways in soups, jams, pickles, salsas and the like. Green tomatoes are known to be rich in ingredients such as tomatine, tomatidine and tomatidenol.

Burdock root is a dicotyledonous Campanula Asteraceae biennial herb, mainly harvested in Japan and grown wild in Europe, Siberia and northeast China. Burdock root contains, as a major ingredient, a fiber such as inulin or cellulose, which is good for the health of the intestine, and contains a great amount of polyphenol compounds, especially isochlorogenic acid, and is thus easily oxidized and discolored when fine slices thereof are left behind. Burdock root is used as an edible ingredient in a variety of foods such as stewed foods, fried foods, pickles and hot pots.

Chickpea is a dicotyledonous perennial herb of the family Rosaceae that is named because it resembles a chick's head, and is grown as an important edible crop in India, Africa, Central America and South America. Chickpeas have a flavor similar to chestnuts, are more aromatic and sweeter when chewed more, are rich in calcium, vitamin C, iron and the like, and are thus known to improve anemia and strengthen the immune system. Chickpea is used not only as a main ingredient in soups, curry and salads, but also as a variety of edible materials.

In the cosmetic composition for skin whitening according to the present invention, the green tomato extract, the burdock root extract and the chickpea extract are preferably obtained by a solvent extraction method, which is extraction through addition of water or an organic solvent, or an ultra-high-pressure extraction method. In this case, the organic solvent is, for example, an extraction solvent selected from the group consisting of anhydrous or aqueous lower alcohol having 1 to 4 carbon atoms, propylene glycol, butylene glycol, glycerin, acetone, ethyl acetate, chloroform, butyl acetate, diethyl ether, dichloromethane, hexane and a mixture thereof.

In the cosmetic composition for skin whitening according to the present invention, the green tomato extract was washed with running water and then dried. To 100 g of the dried green tomato, 1,070 mL, which corresponds to 10.7 times of the weight of tomato, of water was added, followed by extraction at 60° C. for 24 hours. The burdock root extract and chickpea extract were also obtained by conducting extraction in the same manner as for the green tomatoes. After extraction, the extract was centrifuged and filtered to remove impurities, lyophilized, and used for experiments.

In the cosmetic composition for skin whitening according to the present invention, the combination of the green tomato extract, the burdock root extract and the chickpea extract may inhibit the production of melanin in cells.

In the cosmetic composition for skin whitening according to the present invention, the combination including the green tomato extract, the burdock root extract and the chickpea extract may inhibit tyrosinase enzyme activity.

In the cosmetic composition for skin whitening according to the present invention, the combination including the green tomato extract, the burdock root extract and the chickpea extract may inhibit DOPA oxidation.

In the cosmetic composition for skin whitening according to the present invention, the cosmetic composition may be, for example, selected from a solution, a suspension, an emulsion, a paste, cosmetic water, a gel, a water-soluble liquid, a cream, an essence, a surfactant-containing cleanser, an oil, a skin care cosmetic formulation selected from oil-in-water (O/W) and water-in-oil (W/O) formulations; a skin toner; a lotion; an eye cream; a soothing gel; an ointment; a formulation for mask packs; a formulation for body wash; a peeling gel; oil-in-water and water-in-oil makeup bases; a foundation; a skin cover; a makeup cosmetic formulation selected from a lipstick, a lip gloss, a face powder, a two-way cake, an eye shadow, a cheek color and an eyebrow pencil; and a formulation for scalp.

In addition, the cosmetic composition of the present invention can be used in combination with cosmetic compositions other than that of the present invention. In addition, the cosmetic composition according to the present invention may be used according to a conventional method of use, and the number of applications may vary depending on the skin condition or preference of the user.

Hereinafter, the present invention will be described in more detail with reference to the following Examples or Experimental Examples. However, the scope of the present invention is not limited to these Examples or Experimental Examples and also includes modifications equivalent thereto.

### Preparation Example: Preparation of Green Tomato Extract, Burdock Root Extract, and Chickpea Extract of Present Invention

In this example, a green tomato extract, a burdock root extract and a chickpea extract were prepared.

The green tomato extract was washed with running water and then dried. 1,070 mL, which corresponds to 10.7 times of the weight of tomato, of water was added, followed by extraction at 60° C. for 24 hours. The burdock root extract and the chickpea extract were also obtained by conducting extraction in the same manner as for the green tomatoes. After extraction, the extract was centrifuged and filtered to remove impurities, lyophilized, and used for experiments.

In the following, the experiment was conducted to determine the effect of the green tomato extract, burdock root extract and chickpea extract obtained in Preparation Example above on skin whitening.

### Experimental Example 1: Determination of Skin-Whitening Activity of Each Extract

In this experimental example, the effect of the green tomato extract, burdock root extract and chickpea extract prepared in Preparation Example on skin-whitening-activity-related factors was determined.

The skin color is determined by the amount of melanin, which is a dark brown pigment present in the basal layer of the epidermis, which is an important factor in skin whitening. Melanin is biosynthesized in the basal layer of the skin, is transported to keratinocytes, and migrates to the outer layer of the skin by keratinization. Melanin is produced by a series of enzymatic reactions of tyrosinase, tyrosinase-related protein-1 (TRP-1), tyrosinase-related protein-2 (TRP-2) and the like.

Tyrosinase is an enzyme that catalyzes the initial stage of melanin biosynthesis, which is a step of hydroxylating tyrosine to form dihydroxyphenylalanine (DOPA), and further oxidizing the dihydroxyphenylalanine to form dopaquinone. The activity of this enzyme has a significant effect on the production of melanin. Therefore, the following experiment was conducted to identify the effect of each extract on melanin and its related factors that determine the skin-whitening activity.

1) DOPA Oxidation Inhibitory Activity of Green Tomato Extract

The experiment was performed to determine the effect of the green tomato extract obtained in Preparation Example on the oxidation activity of dihydroxyphenylalanine (DOPA) produced in the first step of melanin biosynthesis.

60 μL of a 0.1M phosphate buffer, 60 μL of 5 mM DOPA, 150 μL of the green tomato extract diluted at different concentrations, and 30 μL of 250 unit/mL tyrosinase were mixed and reacted at 37° C. for 5 minutes. Then, absorbance at 475 nm was measured and the result was determined.

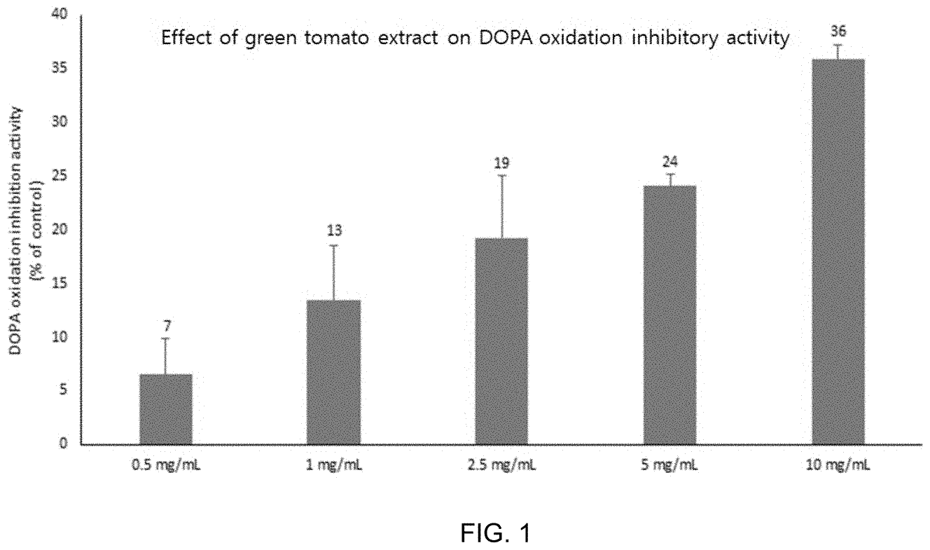

The effect of the green tomato extract on the DOPA oxidation inhibitory activity was observed. The result showed that the DOPA oxidation inhibitory activity of the green tomato extract increased in a concentration-dependent manner, as shown in FIG. 1.

2) Intracellular Tyrosinase Inhibitory Activity of Chickpea Extract

The effect of the chickpea extract of Preparation Example on the intracellular tyrosinase enzyme present in B16/F10 cells, which are Mus musculus skin melanoma, were determined.

The B16/F10 cells were cultured at 37° C. and 5% CO₂ in Dulbecco's modified eagle's medium (DMEM) containing 10% fetal bovine serum (FBS) and 1% penicillin/streptomycin (PEST). When a sufficient amount of cells were cultured, the cells were seeded at 1×10⁵ cells/mL on a 6-well plate and cultured for 2 days. The chickpea extract was diluted to concentrations of 0.5 mg/mL and 1 mg/mL in DMEM medium containing 1% PEST, and then the cells were treated with the chickpea extract and cultured for 3 days. After culturing for 3 days, the cells were collected using a cell scraper and centrifuged at 5,000 rpm and 4° C. for 10 minutes to obtain a cell pellet. For lysis of the cell pellet, 200 μL of PBS containing 1% triton X-100 was added, and the cells were released using a pipette and then frozen and thawed three times to complete cell lysis. After cell lysis, centrifugation was performed at 12,000 rpm and 4° C. for 10 minutes to collect the supernatant to thereby obtain a cell lysate. 100 μL of 0.1M phosphate buffer, 40 μL of 2.5 mM DOPA, and 60 μL of the cell lysate were added to a 96-well plate and reacted at 37° C. for 1 hour, and then absorbance was measured at 475 nm.

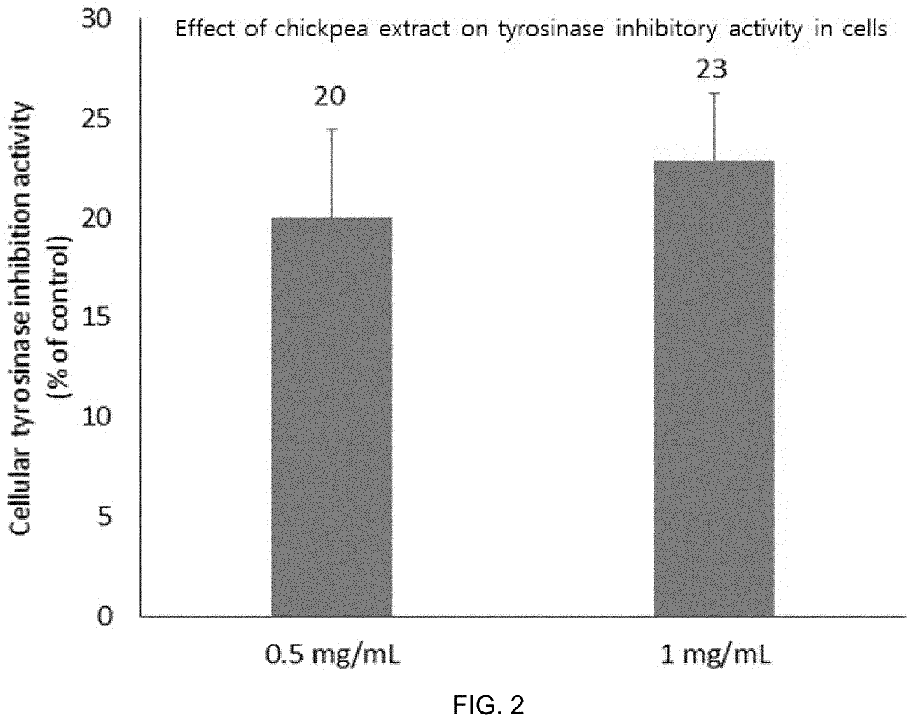

The effect of the chickpea extract on tyrosinase inhibitory activity was observed. The result showed that the tyrosinase inhibitory activity of the chickpea extract increased in a concentration-dependent manner, as shown in FIG. 2.

3) Effect of Burdock Root Extract on Melanin Production in Cells

The effect of the burdock root extract of Preparation Example on intracellular melanin production in B16/F10 cells, which are Mus musculus skin melanoma, was determined.

The B16/F10 cells were cultured at 37° C. and 5% CO₂ in Dulbecco's modified eagle's medium (DMEM) containing 10% fetal bovine serum (FBS) and 1% penicillin/streptomycin (PEST). When a sufficient amount of cells were cultured, the cells were seeded at 1×10⁵ cells/mL on a 6-well plate and cultured for 2 days. The burdock root extract was diluted to concentrations of 0.5 mg/mL and 1 mg/mL in DMEM medium containing 1% PEST, and then the cells were treated with the burdock root extract and cultured for 3 days. After culturing for 3 days, the cells were collected using a cell scraper and centrifuged at 5,000 rpm and 4° C. for 10 minutes to obtain a cell pellet. 200 uL of 1N NaOH was added to the cell pellet, and the cells were released at 60° C. for 24 hours and centrifuged at 12,000 rpm and 4° C. for 10 minutes to collect a supernatant. Then, the supernatant was divided into 100 μL portions on a 96-well plate and then absorbance was measured at 475 nm.

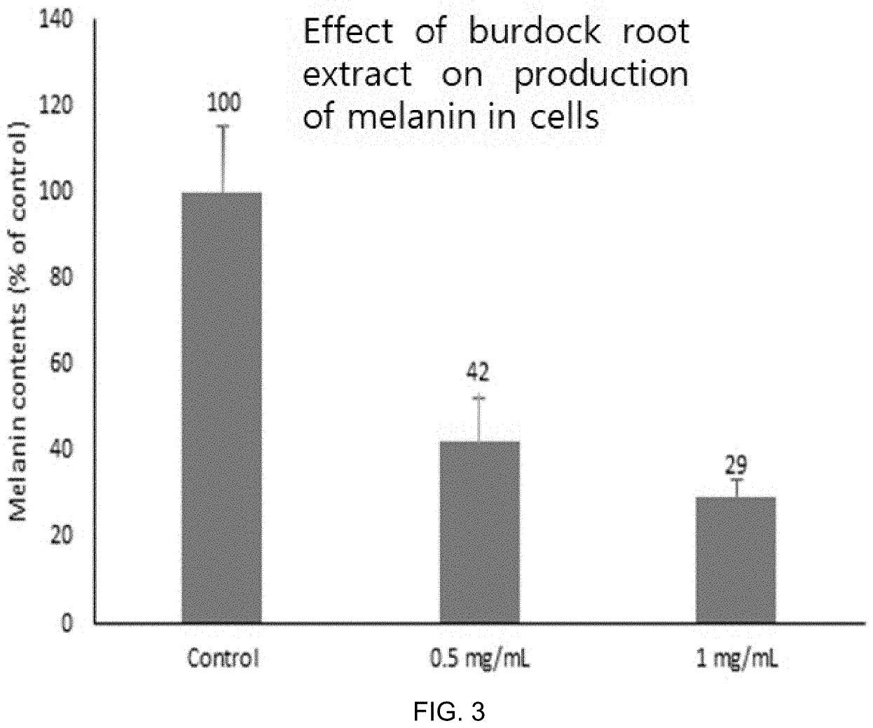

The effect of the burdock root extract on the intracellular melanin production was determined. The result showed that intracellular melanin production was greatly inhibited by increasing the concentration of burdock root extract, as shown in FIG. 3.

The green tomato extract inhibits the oxidative activity of DOPA, which is a whitening-activity-related factor, produced during the first step of melanin biosynthesis, the chickpea extract inhibits tyrosinase activity in cells, and the burdock root extract inhibits melanin production in cells. These results indicate that each extract is useful as an ingredient effective in skin-whitening activity, and a combination of respective extracts was prepared as described below and whether or not the combination had a skin-whitening effect was determined.

### Example: Preparation of Green Tomato Extract, Burdock Root Extract and Chickpea Extract

In this example, the green tomato extract, burdock root extract and chickpea extract prepared in Preparation Example were combined at different mixing ratios to prepare combinations.

These mixing ratios are shown in Table 1 below.

TABLE 1

| Type | Mixing ratio |
|---|---|
| Green | 1:1:1 |
| tomato:burdock:chickpea | 1.5:1:1 |
| | 2:1:1 |

### Experimental Example 2: Determination of Skin-Whitening Activity of Green Tomato Extract, Burdock Root Extract and Chickpea Extract

In this experimental example, the effect of the green tomato extract, burdock root extract and chickpea extract prepared in Preparation Example on skin-whitening-activity-related factors was determined.

1) Intracellular Tyrosinase Inhibitory Activity of Combination

The effects of the green tomato extract, burdock root extract and chickpea extract of Preparation Example on the intracellular tyrosinase enzyme present in B16/F10 cells, which are Mus musculus skin melanoma, were determined.

The B16/F10 cells were cultured at 37° C. and 5% CO₂ in Dulbecco's modified eagle's medium (DMEM) containing 10% fetal bovine serum (FBS) and 1% penicillin/streptomycin (PEST). When a sufficient amount of cells were cultured, the cells were seeded at 1×10⁵ cells/mL on a 6-well plate and cultured for 2 days. A material containing “green tomato:burdock:chickpea” at three mixing ratios of 1:1:1, 1.5:1:1 and 2:1:1, was diluted to concentrations of 0.5 mL/mL, 0.75 mg/mL and 1 mg/mL in DMEM medium containing 1% PEST, and then the cells were treated with the material and cultured for 3 days. After culturing for 3 days, the cells were collected using a cell scraper and centrifuged at 5,000 rpm and 4° C. for 10 minutes to obtain a cell pellet. For lysis of the cell pellet, 200 μL of PBS containing 1% triton X-100 was added, and the cells were released using a pipette and then frozen and thawed three times to complete cell lysis. After cell lysis, centrifugation was performed at 12,000 rpm and 4° C. for 10 minutes to collect the supernatant to thereby obtain a cell lysate. 100 μL of 0.1M phosphate buffer, 40 μL of 2.5 mM DOPA, and 60 μL of the cell lysate were added to a 96-well plate and reacted at 37° C. for 1 hour, and then absorbance was measured at 475 nm.

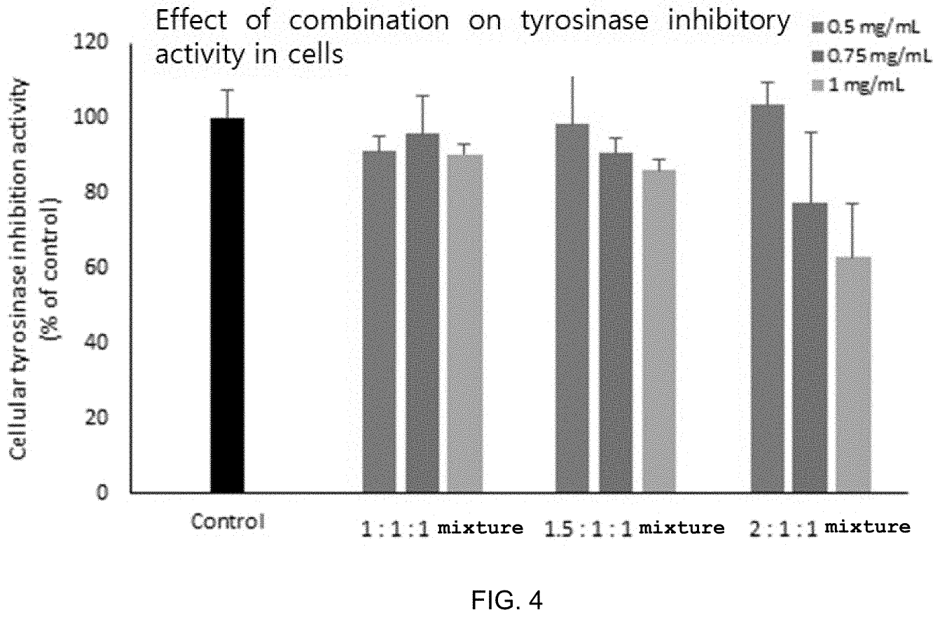

The effect of the combination on tyrosinase inhibitory activity was observed. The result showed that the tyrosinase inhibitory activity of the combination having a concentration of 0.5 mg/mL and a mixing ratio of 2:1:1 increased in a concentration-dependent manner, as shown in FIG. 4.

2) Inhibitory Activity of Melanin Production in Cells of Combination

The effects of the green tomato extract, burdock root extract and chickpea extract of Preparation Example on the production of melanin in B16/F10 cells, which are Mus musculus skin melanoma, were determined.

The B16/F10 cells were cultured at 37° C. and 5% CO₂ in Dulbecco's modified eagle's medium (DMEM) containing 10% fetal bovine serum (FBS) and 1% penicillin/streptomycin (PEST). When a sufficient amount of cells were cultured, the cells were seeded at 1×10⁵ cells/mL on a 6-well plate and cultured for 2 days. A material containing “green tomato:burdock:chickpea” at three mixing ratios of 1:1:1, 1.5:1:1 and 2:1:1, was diluted to concentrations of 0.5 mL/mL, 0.75 mg/mL and 1 mg/mL in DMEM medium containing 1% PEST, and then the cells were treated with the material and cultured for 3 days. After culturing for 3 days, the cells were collected using a cell scraper and centrifuged at 5,000 rpm and 4° C. for 10 minutes to obtain a cell pellet. 200 μL of 1N NaOH was added to the cell pellet, and the cells were lysed at 60° C. for 24 hours and centrifuged at 12,000 rpm and 4° C. for 10 minutes. The supernatant was divided into 200 μL portions on a 96-well plate and then absorbance was measured at 490 nm.

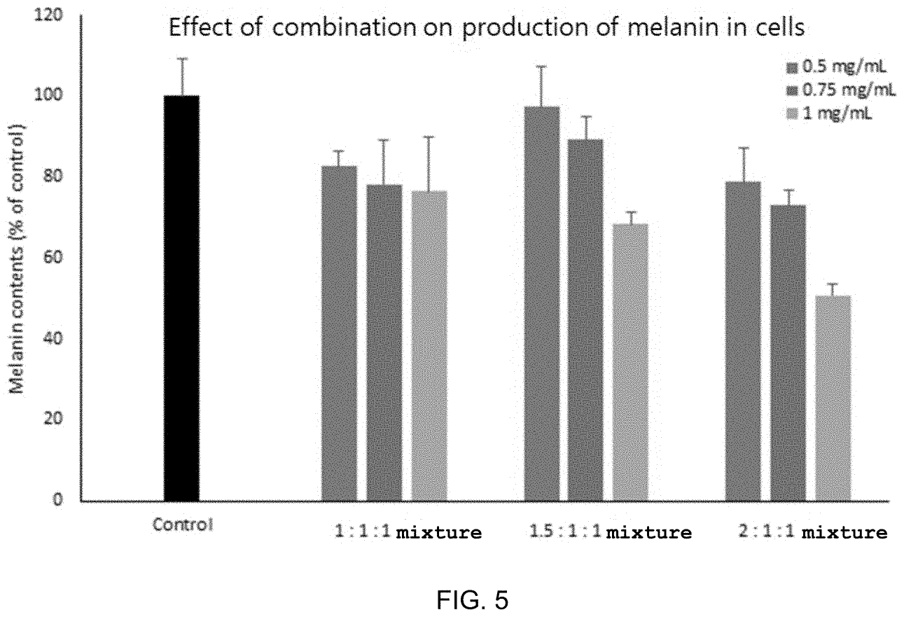

The effect of the combination on the intracellular melanin production was determined. The result showed that the intracellular melanin production was significantly suppressed by treating with an increased concentration of the combination, regardless of the mixing ratio of the combination, as shown in FIG. 5. In particular, the combination having a mixing ratio of 2:1:1 exhibited a great melanin suppression effect.

Therefore, the following experiment was conducted using a combination including “green tomato:burdock:chickpea” in a ratio of 2:1:1, which exhibited significant effects of increasing intracellular tyrosinase inhibitory activity and inhibiting melanin production.

3) Tyrosinase Inhibitory Activity of Combination

An experiment was conducted to determine the effect of the combination of “green tomato:burdock:chickpea” in a ratio of “2:1:1”, among the combinations of the green tomato extract, the burdock root extract and the chickpea extract of Example, on tyrosinase inhibitory activity.

220 μL of 0.1 M phosphate buffer, 40 μL of 1.5 mM tyrosine, 20 μL of a combination including “green tomato:burdock:chickpea” in a mixing ratio of 2:1:1 and diluted at different concentrations, and 20 μL of 1,000 unit/mL tyrosinase were mixed and reacted at 37° C. for 5 minutes, absorbance was measured at 475 nm, and the result was determined.

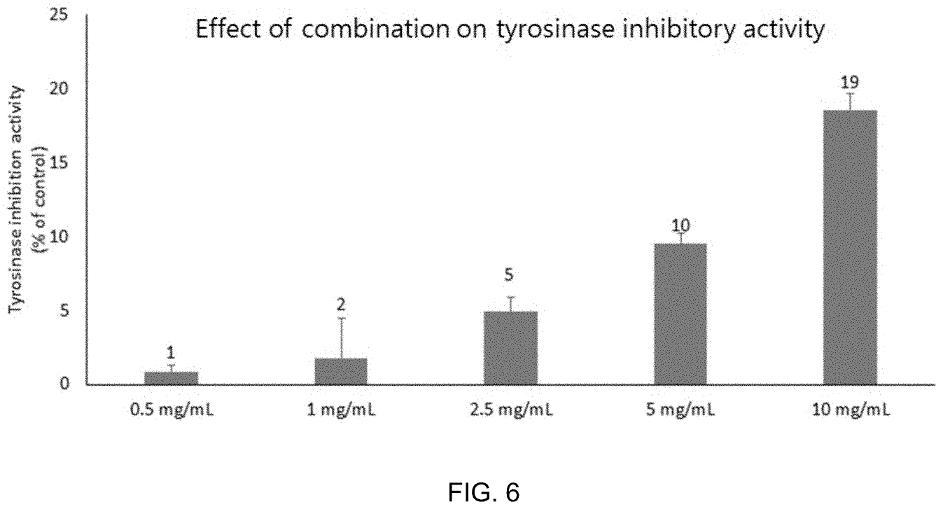

The effect of the combination on tyrosinase inhibitory activity was observed. The result showed that the tyrosinase inhibitory activity of the combination increased in a concentration-dependent manner, as shown in FIG. 6.

4) DOPA Oxidation Inhibitory Activity of Combination

An experiment was conducted to determine the effect of the combination of “green tomato:burdock:chickpea” in a ratio of “2:1:1”, among the combinations of the green tomato extract, the burdock root extract and the chickpea extract of Example on the inhibitory activity of oxidation of DOPA, produced in the first step of melanin biosynthesis.

60 μL of 0.1 M phosphate buffer, 60 μL of 5 mM DOPA, 150 μL of a combination including “green tomato:burdock:chickpea” in a mixing ratio of 2:1:1 and diluted at different concentrations and 30 μL of 250 unit/mL tyrosinase were mixed and reacted at 37° C. for 5 minutes, absorbance was measured at 475 nm and the result was determined.

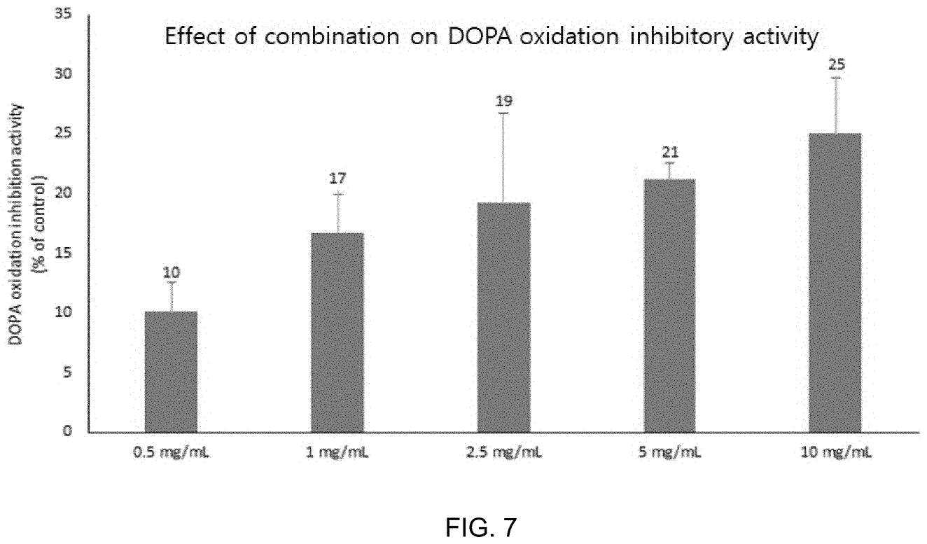

The effect of the combination on DOPA oxidation inhibitory activity was observed. The result showed that the DOPA oxidation inhibitory activity of the combination increased in a concentration-dependent manner, as shown in FIG. 7.

The cosmetic composition according to the present invention contains a combination of a green tomato extract, a burdock root extract and a chickpea extract, thus being effective in inhibiting intracellular melanin production, inhibiting tyrosinase activity, inhibiting DOPA oxidation and thereby providing skin whitening. Therefore, the combination of the green tomato extract, the burdock root extract, and the chickpea extract according to the present invention can be used as raw materials for whitening cosmetic products.

As is apparent from the above description, the present invention provides a cosmetic composition that contains, as an active ingredient, a combination including a green tomato extract, a burdock root extract and a chickpea extract and thereby has an excellent skin-whitening effect.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A cosmetic composition for skin whitening comprising, as an active ingredient, a combination of a green tomato extract, a burdock root extract and a chickpea extract.

2. The cosmetic composition according to claim 1, wherein the combination of the green tomato extract, the burdock root extract and the chickpea extract inhibits production of melanin in cells.

3. The cosmetic composition according to claim 1, wherein the combination of the green tomato extract, the burdock root extract and the chickpea extract inhibits tyrosinase enzyme activity.

4. The cosmetic composition according to claim 1, wherein the combination of the green tomato extract, the burdock root extract and the chickpea extract inhibits DOPA oxidation.
